(19) United States
(12) Patent Application Publication
(10) Pub. No.: US 2025/0177896 A1
(43) Pub. Date: Jun. 5, 2025

(54) FILTER MATERIAL, AIR FILTER, AIR CONDITIONER, WATER FILTER, AND WATER CLEANER

(71) Applicant: SHARP KABUSHIKI KAISHA, Sakai City, Osaka (JP)

(72) Inventors: HIDEOMI YUI, Sakai City, Osaka (JP); TSUYOSHI KAMADA, Sakai City, Osaka (JP); SHIGERU AOMORI, Sakai City, Osaka (JP); YUKA UTSUMI, Sakai City, Osaka (JP)

(21) Appl. No.: 18/843,290

(22) PCT Filed: Dec. 28, 2022
(86) PCT No.: PCT/JP2022/048437

§ 371 Date: Sep. 2, 2024

(30) Foreign Application Priority Data
Mar. 4, 2022 (JP) 2022-033233

(51) Int. Cl. B01D 39/06

(52) U.S. Cl. CPC B01D 39/06

(57) ABSTRACT

Provided are a filter, an air filter, an air conditioner, a water filter, and a water cleaner that have a high antimicrobial property. A filter material is provided with a fiber assembly including a plurality of fibers with protrusions. Each of the plurality of fibers with protrusions includes a fiber having a surface, and a plurality of protrusions arranged on the surface, having a plate shape, and made of aluminum oxide.

### TECHNICAL FIELD

The present disclosure relates to a filter material, an air filter, an air conditioner, a water filter, and a water cleaner. The present application claims priority from Japanese Application No. JP2022-33233, filed on Mar. 4, 2022, the content of which is hereby incorporated by reference into this application.

### BACKGROUND ART

Patent document 1 discloses a filter for air purification. This air-purification filter is provided with the following: a fiber assembly in the form of a nonwoven cloth composed of aluminum fibers or aluminum alloy fibers; a shape retainer made of aluminum or aluminum alloy, having a plurality of holes, covering both flat surfaces of the fiber assembly; a recess formed by roughening at least the surface of the upstream fibers of the fiber assembly; and the skin layer of an alumite layer or a boehmite layer formed on the outer surface of the shape retainer and on the outer surface of the fiber assembly exposed from the holes of the shape retainer (paragraph [0006]).

### CITATION LIST

### Patent Literature

Patent Literature 1: Japanese Patent No. 6433916

### SUMMARY OF INVENTION

### Technical Problem

The air-purification filter disclosed in Patent Literature 1 does not have a sufficient antimicrobial property, a sufficient antivirus property, or others in some cases.

The present disclosure has been made in view of this problem. One aspect of the present disclosure aims to provide a filter, an air filter, an air conditioner, a water filter, and a water cleaner that have a high antimicrobial property.

### Solution to Problem

A filter material according to one aspect of the present disclosure is provided with a fiber assembly including a plurality of fibers with protrusions. Each of the fibers with protrusions includes a fiber having a surface, and a plurality of protrusions arranged on the surface, having a plate shape, and made of aluminum oxide.

An air filter according to another aspect of the present disclosure is provided with the filter material according to the aspect of the present disclosure.

An air conditioner according to another aspect of the present disclosure is provided with the air filter according to the other aspect of the present disclosure.

A water filter according to another aspect of the present disclosure is provided with the filter material according to the aspect of the present disclosure.

A water cleaner according to another aspect of the present disclosure is provided with the water filter according to the other aspect of the present disclosure.

### BRIEF DESCRIPTION OF DRAWINGS

FIG. 1 is a schematic plan view of a filter material according to a first embodiment.

FIG. 2 is a schematic cross-sectional view of a fiber with protrusions provided in the filter material according to the first embodiment.

FIG. 3 is a schematic cross-sectional view of a fiber with protrusions, which is provided in the filter material according to the first embodiment, and a microorganism adhered to the fiber with protrusions.

FIG. 4 is an electron microscopic image of the fiber with protrusions, which is provided in the filter material according to the first embodiment, and a coli bacterium adhered to the fiber with protrusions.

FIG. 5 is an electron microscopic image of the fiber with protrusions, which is provided in the filter material according to the first embodiment, and a coli bacterium adhered to the fiber with protrusions.

FIG. 6 is a flowchart showing a process for manufacturing the filter material according to the first embodiment.

FIG. 7 is a schematic cross-sectional view of a fiber with protrusions provided in a filter material according to a second embodiment.

FIG. 8 is an electron microscopic image of the fiber with protrusions, which is provided in the filter material according to the second embodiment.

FIG. 9 shows the magnitude relationship between the average spacing of the distal ends of a plurality of protrusions, which are provided in the filter material according to the second embodiment, and the sizes of a bacterium and a virus.

FIG. 10 is a schematic cross-sectional view of a fiber with protrusions provided in a filter material according to a third embodiment.

FIG. 11 is an electron microscopic image of the fiber with protrusions, which is provided in the filter material according to the third embodiment.

FIG. 12 shows the magnitude relationship between the average spacing of the distal ends of a plurality of protrusions, which are provided in the filter material according to the third embodiment, and the sizes of a bacterium and a virus.

FIG. 13 schematically illustrates a filter material according to a fourth embodiment.

FIG. 14 schematically illustrates a filter material according to an example of the fourth embodiment.

FIG. 15 schematically illustrates a filter material according to a fifth embodiment.

FIG. 16 schematically illustrates a filter material according to an example of the fifth embodiment.

FIG. 17 schematically illustrates a filter material according to a sixth embodiment.

FIG. 18 schematically illustrates a filter material according to an example of the sixth embodiment.

FIG. 19 schematically illustrates a filter material according to another example of the sixth embodiment.

FIG. 20 is a schematic perspective view of an air filter according to a seventh embodiment.

FIG. 21 is a schematic perspective view of an air conditioner according to an eighth embodiment.

FIG. 22 schematically illustrates a water cleaner according to a ninth embodiment.

### DESCRIPTION OF EMBODIMENTS

Embodiments of the present disclosure will be described with reference to the drawings. It is noted that identical or equivalent components will be denoted by the same signs throughout the drawings, and the description of redundancies will be omitted.

### 1. First Embodiment

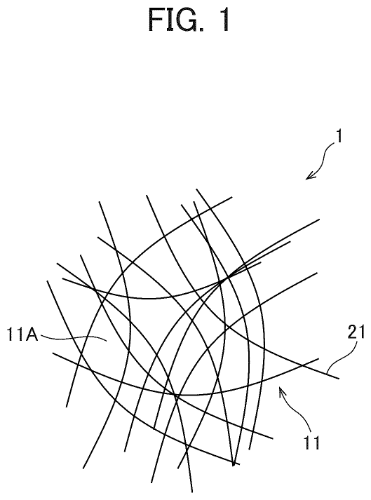

FIG. 1 is a schematic plan view of a filter material according to a first embodiment.

The filter material, 1, illustrated in FIG. 1 according to the first embodiment passes a fluid and deactivates microorganisms contained in a fluid that is to be passed. The fluid that is to be passed is water, air, or other things. The microorganisms that are to be deactivated are bacteria, viruses, and other things.

As illustrated in FIG. 1, the filter material 1 is provided with a fiber assembly 11. The fiber assembly 11 includes a plurality of fibers 21 with projections.

The plurality of fibers 21 with protrusions are intertangled. Between the fibers 21 with protrusions is a space 11A through which a fluid passes.

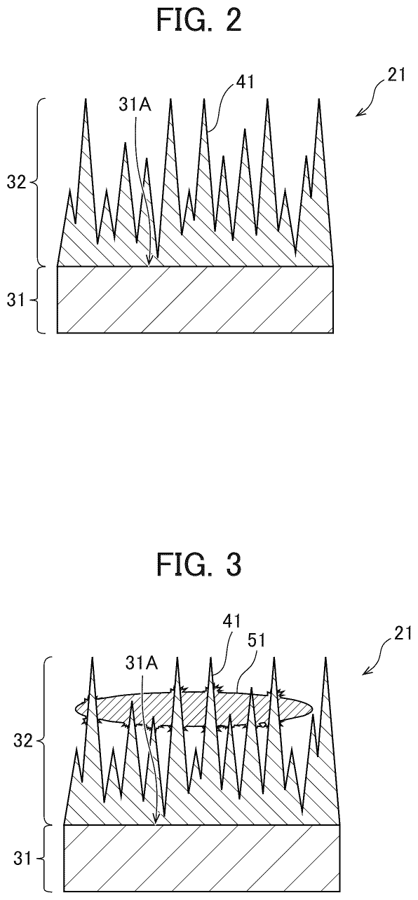

FIG. 2 is a schematic cross-sectional view of a fiber with protrusions provided in the filter material according to the first embodiment. FIG. 2 illustrates a part of the surface layer of a fiber 31, and a protrusion structure 32 formed on the surface layer; FIGS. 3, 7, and 10 illustrate them similarly.

As illustrated in FIG. 2, each protrusion 21 with protrusions includes the fiber 31 and the protrusion structure 32. The protrusion structure 32 has a plurality of projections 41.

The fiber 31 may be either a chemical fiber or a natural fiber. The substance constituting the chemical fiber may be either an organic substance or an inorganic substance. The organic substance is polyester, polyamide, polyethylene, polypropylene, or other things. The inorganic substance is metal, glass, or other things. The metal is aluminum, aluminum alloy, stainless steel, or other things. The natural fiber is cotton, linen, silk, or other things.

The plurality of protrusions 41 are arranged on a surface 31A of the fiber 31. The plurality of protrusions 41 overlap one another on the surface 31A. The plurality of protrusions 41 are arranged densely on the entire surface 31A and filled on the surface 31A. Each of the plurality of protrusions 41 has a plate shape. The plate shape is herein a shape with its height and depth being longer than its width. For instance, a single protrusion 41 in FIG. 2 is structured such that the width in the lateral direction of the drawing sheet is smaller than the height direction with respect to the surface 31A of the fiber 31, and the depth direction of the drawing sheet. The plate shape may be either a flat-plate shape or a curved-plate shape.

The plurality of protrusions 41 are made of aluminum oxide. The plurality of protrusions 41 made of aluminum oxide facilitates forming the protrusion structure 32 onto the surface 31A of the fiber 31.

FIG. 3 is a schematic cross-sectional view of a fiber with protrusions, which is provided in the filter material according to the first embodiment, and a microorganism adhered to the fiber with protrusions.

As illustrated in FIG. 3, the protrusion structure 32 physically damages a microorganism 51 adhered to the protrusion structure 32 to deactivate the microorganism 51. As such, the filter material 1 has an antimicrobial property. The damaged microorganism 51 is a bacterium, a virus, or other things. The filter material 1 thus has an antimicrobial property, an antivirus property, and other properties.

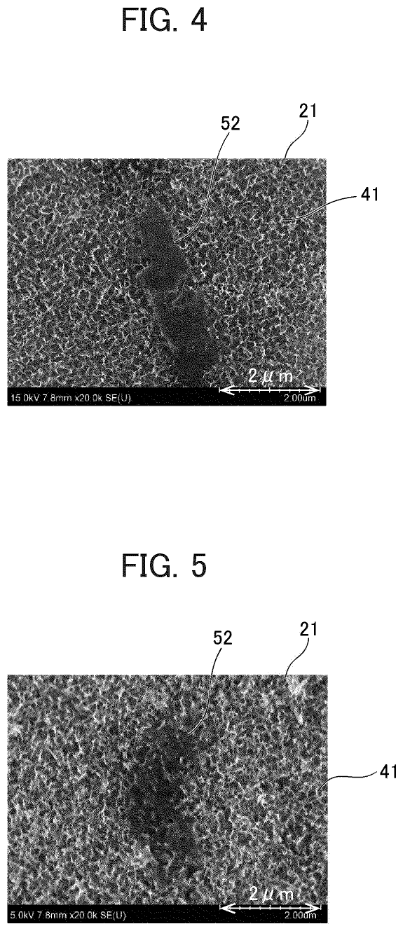

FIG. 4 and FIG. 5 are electron microscopic images of the fiber with protrusions, which is provided in the filter material according to the first embodiment, and a coli bacterium adhered to the fiber with protrusions.

The white portions included in the electron microscope images in FIG. 4 and FIG. 5 are the distal ends of the protrusions 41.

As illustrated in FIG. 4 and FIG. 5, the coli bacterium 52 adhered to the fiber 21 with protrusions is damaged physically and thus no longer retains its original shape. It can be thus understood from FIG. 4 and FIG. 5 that the filter material 1 provided with many fibers 21 with protrusions has an anti-coli-bacterium property.

Each protrusion 41 is a fine protrusion as large as or smaller than the microorganism 51.

The shapes, sizes and orientations of the plurality of protrusions 41 are random. The locations in which the plurality of protrusions 41 are disposed are random.

The adjacent protrusions 41 may overlap each other in the cross section of the fiber 21 with protrusions taken along a line at any site.

The aspect ratio of the height of each protrusion 41 to the thickness of the protrusion 41 is desirably 1 or greater. Accordingly, the protrusion structure 32 physically damages the microorganism 51 easily. This can improve the antimicrobial property of the filter material 1.

Each protrusion 41 is desirably a structure shaped in a sharp blade. Thus, the distal end of each protrusion 41 is desirably thinner than the proximal end of the protrusion 41. Accordingly, the protrusion structure 32 physically damages the microorganism 51 easily. This can improve the antimicrobial property of the filter material 1.

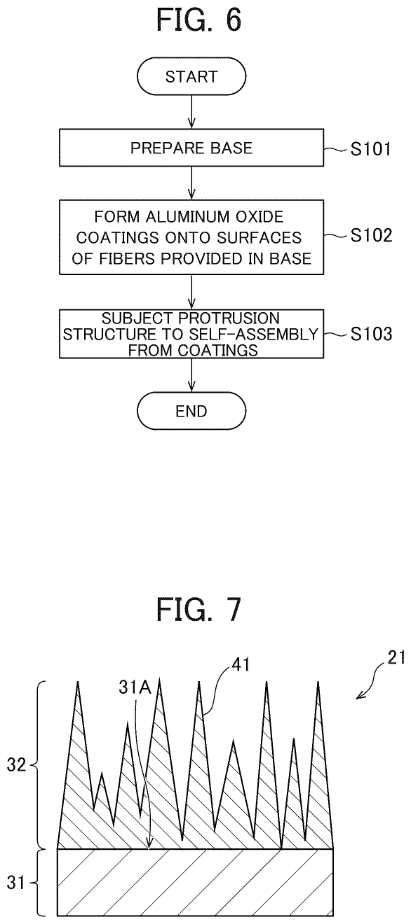

FIG. 6 is a flowchart showing a process for manufacturing the filter material according to the first embodiment.

To manufacture the filter material 1, Steps S101 through S103 shown in FIG. 6 are performed.

Step S101 is preparing a base. The base to be prepared is a fiber assembly including a plurality of fibers 31.

Subsequent Step S102 is forming coatings made of aluminum oxide onto the surfaces 31A of the plurality of fibers 31 provided in the prepared base. This obtains a fiber assembly including a plurality of fibers with coatings. The coatings can be formed through, for example, a sol-gel method using aluminum alkoxide.

Subsequent Step S103 is subjecting the protrusion structure 32 to self-assembly from the formed coatings. The protrusion structure 32 undergoes self-assembly by, for instance, immersing the obtained fiber assembly including the plurality of fibers with coatings into warm water. The temperature of the warm water is 60° C. for instance.

The filter material 1 may be manufactured through another manufacturing method.

### 2. Second Embodiment

The following describes a point in which the second embodiment is different from the first embodiment. With regard to what will not be described, a configuration similar to the configuration used in the first embodiment will be used in the second embodiment as well.

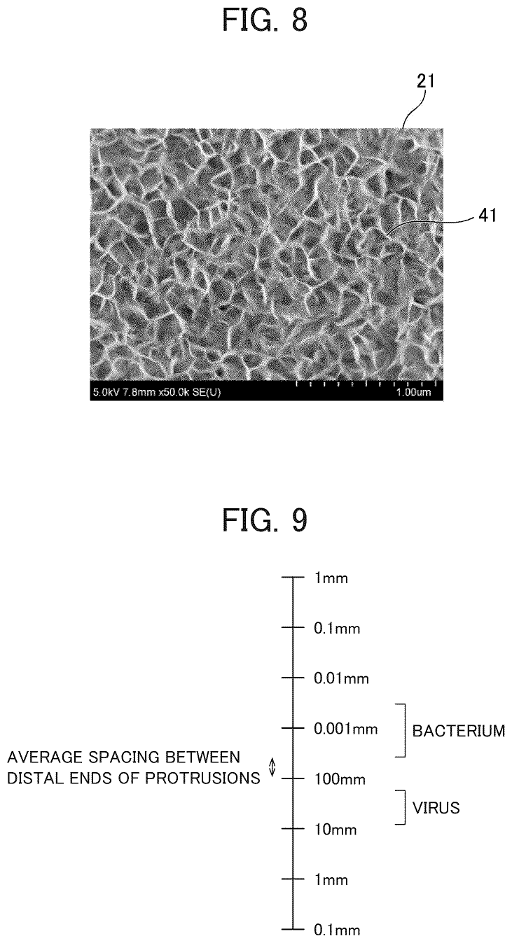

FIG. 7 is a schematic cross-sectional view of a fiber with protrusions provided in the filter material according to the second embodiment. FIG. 8 is an electron microscopic image of the fiber with protrusions, which is provided in the filter material according to the second embodiment.

As illustrated in FIG. 7 and FIG. 8, the distal ends of the plurality of protrusions 41 according to the second embodiment have an average spacing of 100 to 300 nm inclusive.

The average spacing between the distal ends of the plurality of protrusions 41 can be determined by observing, with an electron microscope, the cross section of the fiber 21 with protrusions taken along a line at any site, identifying protrusions 41 having a height of 0.9 times or greater the height of the highest protrusion 41 in the observed field of view, and defining the identified protrusions 41 as targets. For instance, the average spacing can be determined by identifying protrusions 41 having a height of 0.9 times or greater the height of the highest protrusion 41 in any 5-μm long cross section, and measuring the spacing between them. Further, where this average spacing measurement finds at least one site satisfying the average spacing in the fiber 21, it is conceivable in view of the manufacturing method that the fiber 21 has a plurality of sites having such an average spacing. Hence, the fiber 21 satisfying, in at least one site, the condition of the average spacing in the measurement is regarded as being capable of damaging bacteria. Further, it is more desirable that a plurality of given sites be measured in the average spacing measurement, and that the requirement for the average spacing be satisfied in a half or more of these sites. For instance, it is more desirable that ten given sites undergo the average spacing measurement, and that the requirement for the average spacing be satisfied in five or more of the sites, which makes up half or more, because bacteria can be damaged more certainly in such a desirable case.

FIG. 9 shows the magnitude relationship between the average spacing of the distal ends of a plurality of protrusions, which are provided in the filter material according to the second embodiment, and the sizes of a bacterium and a virus.

As illustrated in FIG. 9, bacteria are typically around 0.001-mm (1000-nm) large. Thus, the above-described average spacing between the distal ends of the plurality of protrusions 41, i.e., 100 to 300 nm inclusive, is slightly smaller than the size of bacteria. Accordingly, a bacterium adhered to the fiber 21 with protrusions is physically damaged easily. This can improve the antimicrobial property of the filter material 1.

The average spacing between the distal ends of the plurality of protrusions 41 can be regulated in accordance with the time for immersing the fiber assembly into the warm water in Step S103, and the temperature of the warm water.

### 3. Third Embodiment

The following describes a point in which the third embodiment is different from the first embodiment. With regard to what will not be described, a configuration similar to the configuration used in the first embodiment will be used in the third embodiment as well.

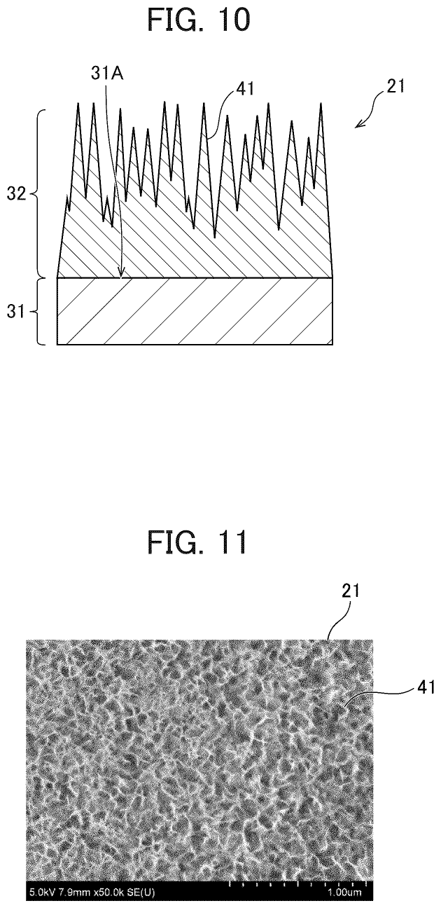

FIG. 10 is a schematic cross-sectional view of a fiber with protrusions provided in a filter material according to the third embodiment. FIG. 11 is an electron microscopic image of the fiber with protrusions, which is provided in the filter material according to the third embodiment.

As illustrated in FIG. 10 and FIG. 11, the distal ends of the plurality of protrusions 41 according to the third embodiment have an average spacing of 10 to 100 nm inclusive.

The average spacing in the third embodiment is determined through a method similar to the method of determining the average spacing in the second embodiment.

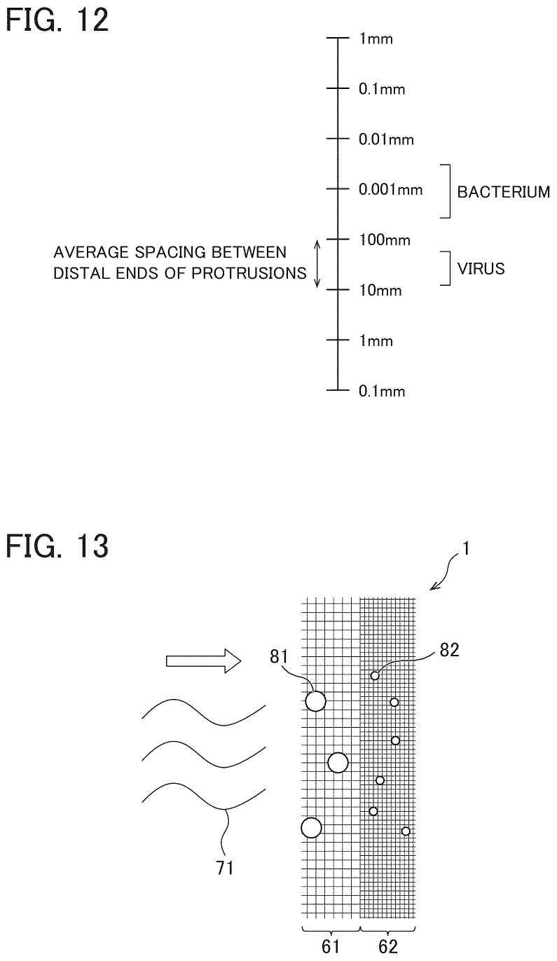

FIG. 12 shows the magnitude relationship between the average spacing of the distal ends of a plurality of protrusions, which are provided in the filter material according to the third embodiment, and the sizes of a bacterium and a virus.

As illustrated in FIG. 12, viruses are typically around 10- to 100-nm large. Thus, the above-described average spacing between the distal ends of the plurality of protrusions 41, i.e., 10 to 100 nm inclusive, is as large as the size of viruses. Accordingly, a virus adhered to the fiber 21 with protrusions is physically damaged easily. This can improve the antivirus property of the filter material 1.

The average spacing between the distal ends of the plurality of protrusions 41 can be regulated in accordance with the time for immersing the fiber assembly into the warm water in Step S103, and the temperature of the warm water.

### 4. Fourth Embodiment

The following describes a point in which the fourth embodiment is different from the first embodiment. With regard to what will not be described, a configuration similar to the configuration used in the first embodiment will be used in the fourth embodiment as well.

FIG. 13 schematically illustrates a filter material according to the fourth embodiment.

As illustrated in FIG. 13, the filter material 1 according to the fourth embodiment is provided with two fiber assemblies 61 and 62. The filter material 1 may be provided with three or more fiber assemblies.

Each of the fiber assemblies 61 and 62 has a layer shape. The fiber assemblies 61 and 62 are stacked. Accordingly, a fluid 71 that is to pass through the filter material 1 passes through the fiber assemblies 61 and 62 sequentially.

The fiber assemblies 61 and 62 include the fiber assembly 11 including the plurality of fibers 21 with protrusions. This can provide the filter material 1 with an antimicrobial property. One fiber assembly 11 may be included, or two or more fiber assemblies 11 may be included.

The fiber assemblies 61 and 62 have mutually different fiber characteristics. The fiber characteristics include at least one selected from the group consisting of a fiber diameter and a weight per unit area. This can make a difference in size between removed targets 81 and 82 that are to be respectively removed by the fiber assemblies 61 and 62. This can prevent the filter material 1 from clogging. Consequently, the filter material 1 can maintain its antimicrobial property for a long period of time.

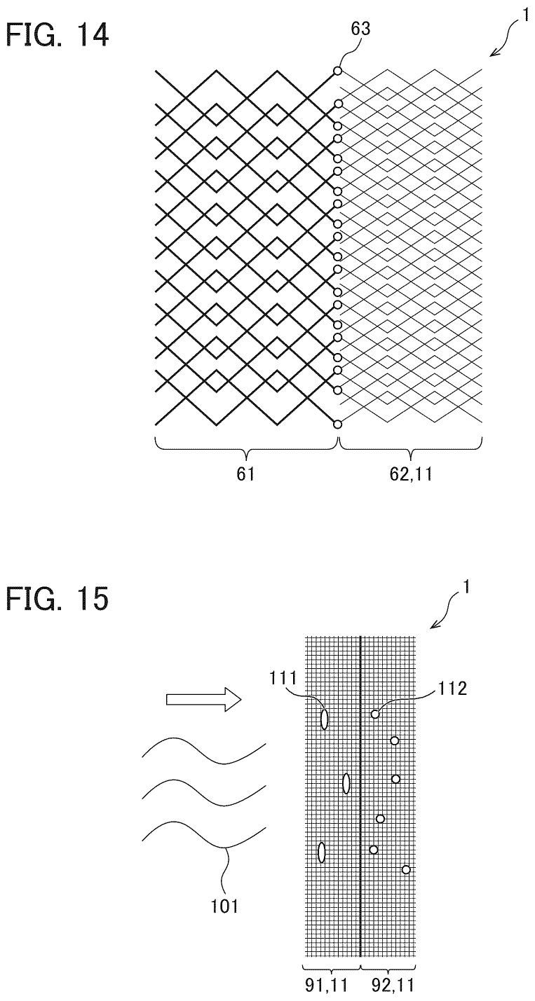

FIG. 14 schematically illustrates a filter material according to an example of the fourth embodiment.

As illustrated in FIG. 14, the filter material 1 according to the example of the fourth embodiment is provided with the fiber assemblies 61 and 62, and a cured adhesive 63.

The fiber assemblies 61 and 62 are joined together with the cured adhesive 63.

A second fiber assembly, 62, has a fiber diameter smaller than the fiber diameter of a first fiber assembly, 61.

The second fiber assembly 62, having a relatively small fiber diameter, is the fiber assembly 11 including the plurality of fibers 21 with protrusions.

The filter material 1 can be manufactured by applying an adhesive to one surface of the first fiber assembly 11, having a relatively large fiber diameter, followed by joining the first fiber assembly 11 and the second fiber assembly 11 together with the applied adhesive, followed by curing the adhesive to transform the adhesive into the cured adhesive 63.

### 5. Fifth Embodiment

The following describes a point in which the fifth embodiment is different from the first embodiment. With regard to what will not be described, a configuration similar to the configuration used in the first embodiment will be used in the fifth embodiment as well.

FIG. 15 schematically illustrates a filter material according to the fifth embodiment.

As illustrated in FIG. 15, the filter material 1 according to the fifth embodiment is provided with two fiber assemblies 91 and 92. The filter material 1 may be provided with three or more fiber assemblies.

Each of the fiber assemblies 91 and 92 has a layer shape. The fiber assemblies 91 and 92 are stacked. Accordingly, a fluid 101 that is to pass through the filter material 1 passes through the fiber assemblies 91 and 92 sequentially.

The fiber assemblies 91 and 92 have the same fiber characteristic. The fiber characteristic includes at least one selected from the group consisting of a fiber diameter and a weight per unit area. The fiber assemblies 91 and 92 may have mutually different fiber characteristics.

Each of the fiber assemblies 91 and 92 is the fiber assembly 11 including the plurality of fibers 21 with protrusions. However, the plurality of fiber assemblies 11 have mutually different average spacings between the distal ends of the plurality of protrusions 41.

This can make a difference in kind between microorganisms 111 and 112 that are to be respectively deactivated by the fiber assemblies 91 and 92. For example, the microorganism 111 can be a bacterium, and the microorganism 112 can be a virus.

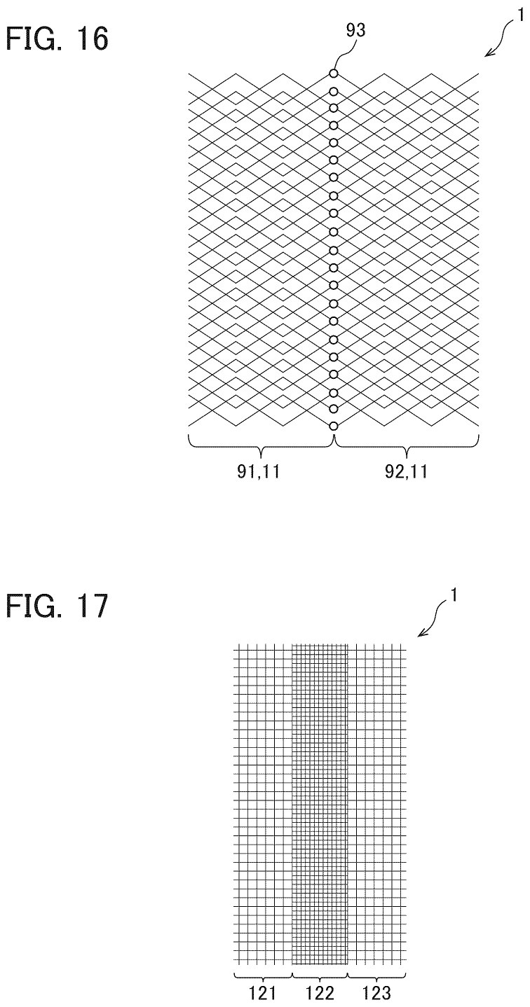

FIG. 16 schematically illustrates a filter material according to an example of the fifth embodiment.

As illustrated in FIG. 16, the filter material 1 according to the example of the fifth embodiment is provided with the fiber assemblies 91 and 92, and a cured adhesive 93.

The fiber assemblies 91 and 92 are joined together with the cured adhesive 93.

Each of the fiber assemblies 91 and 92 is the fiber assembly 11 including the plurality of fibers 21 with protrusions.

A second fiber assembly, 92, has a smaller average spacing between the distal ends of the plurality of protrusions 41 than the average spacing between the distal ends of the plurality of protrusions 41 of a first fiber assembly, 91.

The filter material 1 can be manufactured by applying an adhesive to one surface of one of the first fiber assembly 91 and second fiber assembly 92, followed by joining the first fiber assembly 91 and the second fiber assembly 92 together with the applied adhesive, followed by curing the adhesive to transform the adhesive into the cured adhesive 93.

### 6. Sixth Embodiment

The following describes a point in which the sixth embodiment is different from the first embodiment. With regard to what will not be described, a configuration similar to the configuration used in the first embodiment will be used in the sixth embodiment as well.

FIG. 17 schematically illustrates a filter material according to the sixth embodiment.

As illustrated in FIG. 17, the filter material 1 according to the sixth embodiment is provided with three fiber assemblies 121, 122 and 123. The filter material 1 may be provided with four or more fiber assemblies.

Each of the fiber assemblies 121, 122, and 123 has a layer shape. The fiber assemblies 121, 122, and 123 are stacked. Accordingly, a fluid that is to pass through the filter material 1 passes through the fiber assemblies 121, 122, and 123 sequentially.

The fiber assemblies 121, 122, and 123 include the fiber assembly 11 including the plurality of fibers 21 with protrusions. This can provide the filter material 1 with an antimicrobial property. One fiber assembly 11 may be included, or two or more fiber assemblies 11 may be included.

The fiber assemblies 121, 122, and 123 include the fiber assemblies 121 and 123 having a first fiber characteristic, and the fiber assembly 122 having a second fiber characteristic. The first fiber characteristic and the second fiber characteristic are different from each other. The fiber characteristics include at least one selected from the group consisting of a fiber diameter and a weight per unit area.

The fiber assemblies 121 and 123, having the first fiber characteristic, and the fiber assembly 122, having the second fiber characteristic, are stacked alternately.

The rigidity of the fiber assemblies 121 and 123, having the first fiber characteristic, and the rigidity of the fiber assembly 122, having the second fiber characteristic, are different from each other in some cases. The expansion of the fiber assemblies 121 and 123, having the first fiber characteristic, due to temperature change and the expansion of the fiber assembly 122, having the second fiber characteristic, due to temperature change are different from each other in some cases. The contraction of the fiber assemblies 121 and 123, having the first fiber characteristic, due to temperature change and the contraction of the fiber assembly 122, having the second fiber characteristic, due to temperature change are different from each other in some cases. The foregoing can constitute a cause of deformation in the filter material 1, such as warpage, due to temperature change. However, stacking alternately the fiber assemblies 121 and 123, having the first fiber characteristic, and the fiber assembly 122, having the second fiber characteristic, can prevent the filter material 1 from deformation due to temperature change.

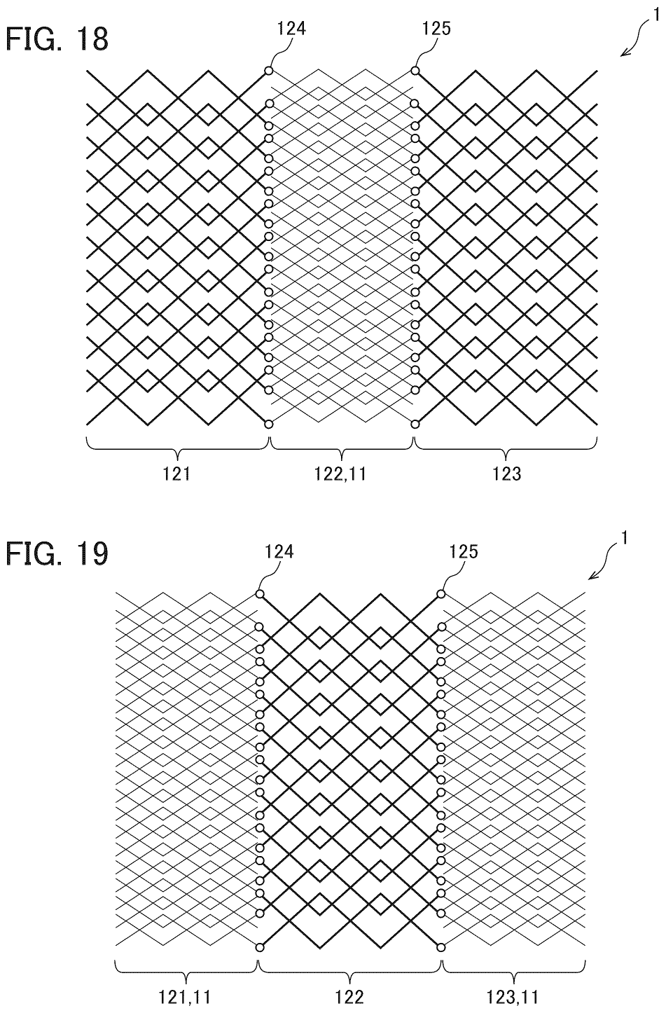

FIG. 18 schematically illustrates a filter material according to an example of the sixth embodiment.

The filter material 1 according to the example of the sixth embodiment is provided with the fiber assemblies 121, 122, and 123, and cured adhesives 124 and 125.

The fiber assemblies 121 and 122 are joined together with the cured adhesive 124. The fiber assemblies 122 and 123 are joined together with the cured adhesive 125.

A second fiber assembly, 122, has a fiber diameter smaller than the fiber diameter of first fiber assemblies, 121 and 123.

The second fiber assembly 122, having a relatively small fiber diameter, is the fiber assembly 11 including the plurality of fibers 21 with protrusions.

The filter material 1 can be manufactured by applying an adhesive to one surface of each of the first fiber assemblies 121 and 123, having a relatively large fiber diameter, followed by joining the fiber assemblies 121 and 122 together with the adhesive applied to the fiber assembly 121, and joining the fiber assemblies 123 and 122 together with the adhesive applied to the fiber assembly 123, followed by transforming the adhesives applied to the fiber assemblies 121 and 123 into the respective cured adhesives 124 and 125.

FIG. 19 schematically illustrates a filter material according to another example of the sixth embodiment.

The following describes a point in which the filter material 1 illustrated in FIG. 19 according to the other example of the sixth embodiment is different from the filter material 1 illustrated in FIG. 18 according to the example of the sixth embodiment.

In the other example of the sixth embodiment, second fiber assemblies, 121 and 123, have a fiber diameter smaller than the fiber diameter of a first fiber assembly, 122.

Each of the second fiber assemblies 121 and 123, having a relatively small fiber diameter, is the fiber assembly 11 including the plurality of fibers 21 with protrusions.

The filter material 1 can be manufactured by applying an adhesive to both surfaces of the first fiber assembly 122, having a relatively large fiber diameter, followed by joining the fiber assemblies 121 and 122 together with the adhesive applied to one of the surfaces of the first fiber assembly 122, and joining the fiber assemblies 123 and 122 together with the adhesive applied to the other surface of the first fiber assembly 122, followed by transforming the adhesives applied to one of the surfaces and the other surface of the first fiber assembly 122 into the respective cured adhesives 124 and 125.

### 7. Seventh Embodiment

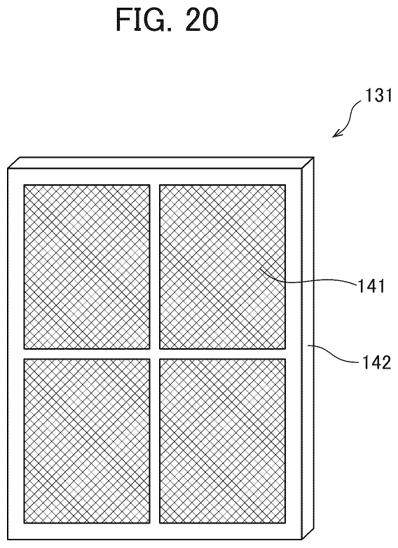

FIG. 20 is a schematic perspective view of an air filter according to a seventh embodiment.

The air filter, 131, illustrated in FIG. 20 according to the seventh embodiment passes air and deactivates the microorganism 51 contained in the air that is to be passed.

As illustrated in FIG. 20, the air filter 131 is provided with a filter material 141 and an outer frame 142. The air filter 131 is provided with an adhesive layer, which is not shown.

The filter material 141 is the filter material 1 according to any one of the first to sixth embodiments.

The outer frame 142 retains the filter material 141.

The adhesive layer fixes the outer frame 142 to the filter material 141.

The outer frame 142 and the adhesive layer may be omitted. Where the outer frame 142 and the adhesive layer are omitted, the outer peripheral surface of the filter material 141 desirably undergoes processing, such as pressing and solidifying the fibers 21 with protrusions, for preventing falling of the fibers 21 with protrusions.

The filter material 141 may undergo charge processing for improving its dust absorbing performance.

### 8. Eighth Embodiment

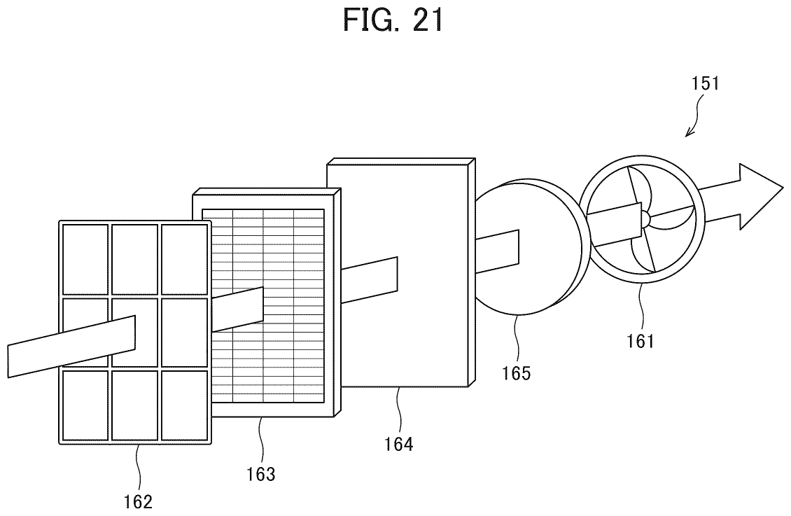

FIG. 21 is a schematic perspective view of an air conditioner according to the eighth embodiment.

The air conditioner, 151, illustrated in FIG. 21 has the function of purifying air and the function of humidifying air. The air conditioner 151 thus operates as an air purifier and humidifier. The air conditioner 151 may function as any thing other than an air purifier and humidifier. For instance, the air conditioner 151 may have, but not limited to, the function of cooling, heating, or dehumidifying air, or the function of supplying ions. The air conditioner 151 may have no humidifying function.

As illustrated in FIG. 21, the air conditioner 151 is provided with an air-blowing fan 161, a prefilter 162, an antimicrobial HEPA filter 163, a deodorant filter 164, and a humidifying filter 165. The air conditioner 151 is provided with an outer frame, which is not shown. In the outer frame, an inlet and an outlet are formed.

The air-blowing fan 161 generates a flow of air that is taken into the inlet, then passes through the prefilter 162, antimicrobial HEPA filter 163, deodorant filter 164, and humidifying filter 165, and is then discharged from the outlet.

The prefilter 162 passes air and removes coarse foreign matters contained in the air that is to be passed from the air.

The antimicrobial HEPA filter 163 passes air and removes dust contained in the air that is to be passed from the air.

The deodorant filter 164 passes air and removes an odor component contained in the air that is to be passed from the air.

The humidifying filter 165 passes air and humidifies the air that is to be passed.

The antimicrobial HEPA filter 163 is provided with the air filter 131 according to the seventh embodiment. This can remove the microorganism 51 contained in the air that is to pass through the antimicrobial HEPA filter 163, thereby deactivating the microorganism 51. Other than the antimicrobial HEPA filter 163, a dust-collecting filter that removes dust may be provided with the air filter 131.

### 9. Ninth Embodiment

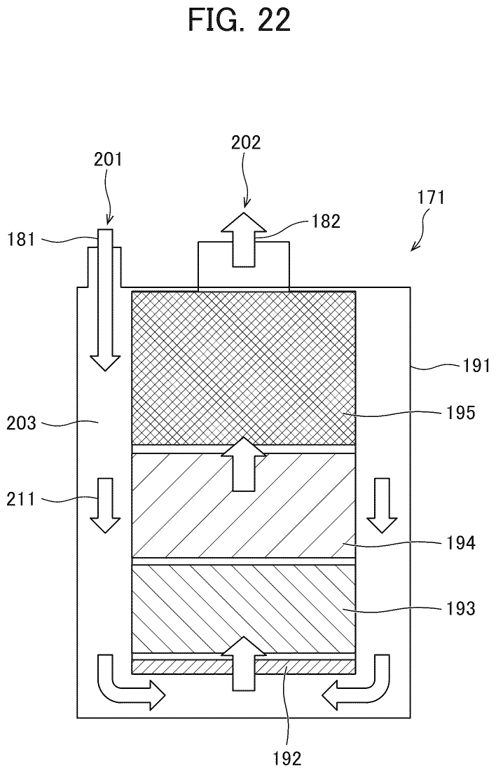

FIG. 22 schematically illustrates a water cleaner according to a ninth embodiment.

The water cleaner, 171, illustrated in FIG. 22 according to the ninth embodiment cleans supplied raw water 181 to generate clean water 182 and supplies the generated clean water 182.

As illustrated in FIG. 22, the water cleaner 171 is provided with a casing 191, a nonwoven cloth 192, an activated carbon layer 193, a ceramic particle layer 194, and an antimicrobial/antivirus layer 195.

The casing 191 incorporates a raw-water inlet 201, a clean-water outlet 202, and a flow channel 203. The flow channel 203 extends from the raw-water inlet 201 to the clean-water outlet 202 and leads water 211 from the raw-water inlet 201 to the clean-water outlet 202.

The nonwoven cloth 192 passes the water 211 and removes dust contained in the water 211 that is to be passed from the water 211.

The activated carbon layer 193 is made of activated carbon. The activated carbon layer 193 passes the water 211 and removes chlorine, organochlorine compounds, and other substances contained in the water 211 that is to be passed from the water 211.

The ceramic particle layer 194 is composed of ceramic particles. The ceramic particle layer 194 passes the water 211 and removes various impurities contained in the water 211 that is to be passed from the water 211.

The antimicrobial/antivirus layer 195 is provided with a water filter including the filter material 1 according to any one of the first to sixth embodiments. The antimicrobial/antivirus layer 195 passes the water 211 and deactivates the microorganism 51 contained in the water 211 that is to be passed.

Typical chemical fibers are hydrophobic. In contrast, the fiber 21 with protrusions, which has an outermost surface made of hydrophilic aluminum oxide, is hydrophilic. This can lower water pressure that is required for the water 211 to pass through the filter material 1 and antimicrobial/antivirus layer 195.

Further, when the microorganism 51 is deactivated by a chemical, such as an antibacterial agent, the chemical can be eluted into the water 211. In contrast, when the microorganism 51 is deactivated by the filter material 1, the microorganism 51 is deactivated physically, so that a component that is undesirable for the water 211 can be prevented from elution.

Further, deactivating the microorganism 51 by the use of a catalyst, a carrier supporting the catalyst, and a light source for irradiating the catalyst with light requires an element, such as the light source, which makes the water cleaner large and complicated. In contrast, deactivating the microorganism 51 by the use of the filter material 1 can prevent the need for such an element.

The present disclosure is not limited to the above-described embodiments. The present disclosure may be replaced with a configuration substantially identical to that described in the above-described embodiments, a configuration that provides the same action and effect, or a configuration that can achieve the same object.

1. A filter material comprising a fiber assembly including a plurality of fibers with protrusions,
wherein each of the plurality of fibers with protrusions includes
a fiber having a surface, and
a plurality of protrusions arranged on the surface, having a plate shape, and made of aluminum oxide.

2. The filter material according to claim 1, wherein a ratio of a height of each of the plurality of protrusions to a thickness of the protrusion is 1 or greater.

3. The filter material according to claim 1, wherein a distal end of each of the plurality of protrusions is thinner than a proximal end of the protrusion.

4. The filter material according to claim 1, wherein distal ends of the plurality of protrusions have an average spacing of 100 to 300 nm inclusive.

5. The filter material according to claim 1, wherein distal ends of the plurality of protrusions have an average spacing of 10 to 100 nm inclusive.

6. The filter material according to claim 1, comprising a plurality of fiber assemblies having mutually different fiber characteristics and being stacked,
wherein the plurality of fiber assemblies include the fiber assembly including the plurality of fibers with protrusions.

7. The filter material according to claim 6, wherein
the plurality of fiber assemblies include a first fiber assembly, and a second fiber assembly having a fiber diameter smaller than a fiber diameter of the first fiber assembly, and
the second fiber assembly is the fiber assembly including the plurality of fibers with protrusions.

8. The filter material according to claim 6, wherein
the plurality of fiber assemblies include a fiber assembly having a first fiber characteristic, and a fiber assembly having a second fiber characteristic different from the first fiber characteristic, and
the fiber assembly having the first fiber characteristic and the fiber assembly having the second fiber characteristic are stacked alternately.

9. The filter material according to claim 1, comprising a plurality of fiber assemblies being stacked,
wherein each of the plurality of fiber assemblies is the fiber assembly including the plurality of fibers with protrusions, and
the plurality of fiber assemblies have mutually different average spacings between distal ends of the plurality of protrusions.

10. An air filter comprising the filter material according to claim 1.

11. An air conditioner comprising the air filter according to claim 10.

12. A water filter comprising the filter material according to claim 1.

13. A water cleaner comprising the water filter according to claim 12.
